(19) United States
(12) Patent Application Publication
(10) Pub. No.: US 2026/0274884 A1
(43) Pub. Date: Sep. 17, 2026

(54) PROCESS FOR EXTRACTION OF ADENOSINE TRIPHOSPHATE (ATP) FROM PLANT MATERIAL

(71) Applicant: Jonathan Davis, New South Wales (AU)

(72) Inventor: Jonathan Davis, New South Wales (AU)

(21) Appl. No.: 19/167,980

(22) PCT Filed: Mar. 24, 2024
(86) PCT No.: PCT/AU2024/050271

§ 371 Date: Sep. 23, 2025

(30) Foreign Application Priority Data
Mar. 23, 2023 (AU) 2023900812
Nov. 8, 2023 (AU) 2023903588

(51) Int. Cl. C07H 19/20; B01D 11/02; C07H 1/08

(52) U.S. Cl. CPC C07H 19/20; B01D 11/028; B01D 11/0288; C07H 1/08

(57) ABSTRACT

Accordingly, in one aspect of the invention there is provided a method of manufacturing an ATP enriched plant based extract comprising: gathering a collection of plant material; saturating the collection of plant material in a solvent to generate a liquid extract; filtering the liquid extract to remove solid plant matter therefrom to form a filtered liquid extract; exposing the filtered liquid extract to UV light; reducing the filtered liquid extract to form a liquid concentrate; and cleaning the liquid concentrate to create an ATP enriched oil extract

### RELATED APPLICATIONS

The present application claims priority from Australian Provisional Patent Application No. 2023900812 filed on 23 Mar. 2023 and Australian Provisional Patent Application no. 2023903588 filed 8 Nov. 2023, the entire contents of which are incorporated herein by reference.

### FIELD OF INVENTION

The present invention relates generally to a process for extracting Adenosine Triphosphate (ATP) from chlorophyl based plant material for treatment of medical conditions.

### BACKGROUND OF THE INVENTION

Adenosine Triphosphate (ATP) is an organic compound found in living organisms and which provides energy to drive and support cells. ATP is made of a nitrogen base (adenine) and a sugar molecule (ribose), which create adenosine, plus three phosphate molecules.

In the human body, molecules held in the fats, proteins, and carbohydrates consumed as food or drink are sources of energy to make ATP. ATP is created in the body through a process called hydrolysis. After food is digested, it becomes synthesized into glucose, which is a form of sugar. Glucose is the main source of fuel that our cells' mitochondria use to convert caloric energy from food into ATP, to be used to supply to the cells. ATP is made via a process called cellular respiration that occurs in the mitochondria of a cell. Mitochondria are tiny subunits within a cell that specialize in extracting energy from the foods we eat and converting it into ATP. Without ATP cells wouldn't be able to use the energy held in food to fuel cellular processes, and an organism would die as a result.

In plants, during photosynthesis, plants capture and store the energy they derive from light in ATP molecules. ATP is produced in the plant's chloroplast's thylakoid membrane. A main problem in extracting ATP from plants is that the compound is very labile and extraction procedures must rupture cells, release the ATP, inactivate normally present ATP'ase enzymes, and preserve the compound in such a state as to prevent hydrolysis. Typically, extraction of ATP from plants requires harvesting parts of the plant and treating the plant with pressurised carbon dioxide or through application of a solvent, to absorb and separate the ATP from the plant parts to form a liquid extract. The liquid extract is then filtered to remove parts of the plant from the liquid at which stage it is then distilled and purified by way of conventional means, to form a concentrated and purified oil containing ATP. This product can then be used directly for a variety of treatments, as required.

A problem with most existing processes for extracting the oil is that they fail to produce ATP to appropriate levels to facilitate optimal use in medical applications.

Thus, there is a need to improve the extraction process to address the limitations of existing extraction processes, as identified above.

The above references to and descriptions of prior proposals or products are not intended to be, and are not to be construed as, statements or admissions of common general knowledge in the art. In particular, the above prior art discussion does not relate to what is commonly or well known by the person skilled in the art, but assists in the understanding of the inventive step of the present invention of which the identification of pertinent prior art proposals is but one part.

### STATEMENT OF INVENTION

The invention according to one or more aspects is as defined in the independent claims. Some optional and/or preferred features of the invention are defined in the dependent claims.

Accordingly, in one aspect of the invention there is provided a method of manufacturing an ATP enriched plant based extract comprising:
gathering a collection of plant material;
saturating the collection of plant material in a solvent to generate a liquid extract;
filtering the liquid extract to remove solid plant matter therefrom to form a filtered liquid extract;
exposing the filtered liquid extract to UV light;
reducing the filtered liquid extract to form a liquid concentrate; and
cleaning the liquid concentrate to create an ATP enriched oil extract

The step of gathering the collection of plant material may comprise drying and/or humidifying the plant material to substantially remove moisture therefrom.

prior to exposing the filtered liquid extract to UV light, the filtered liquid extract is diluted to form a diluted liquid extract.

The step of diluting the filtered liquid extract may comprise adding a solvent to the filtered liquid extract at a ratio of one part liquid extract to two part solvent.

The solvent may be propanol.

In one embodiment, the step of exposing the filtered liquid extract to facilitate the generation of adenosine triphosphate within the filtered liquid extract may comprise exposing the filtered liquid extract to natural UV light.

In another embodiment, the step of exposing the filtered liquid extract to facilitate the generation of adenosine triphosphate within the filtered liquid extract comprises exposing the filtered liquid extract to UV light generated by a UV lamp.

Prior to exposing the filtered liquid extract to UV light, the diluted liquid extract may be placed in a glass jar such that the UV light can irradiate the filtered liquid extract contained therein.

The step of reducing the filtered liquid extract to form a liquid concentrate may comprise placing the filtered liquid extract into a still and reduced the filtered liquid extract using an induction plate.

The heat applied by the induction plate may be able to safely reduce the filtered liquid extract to the liquid concentrate and the heat applied may cause decarboxylation to occur within the liquid concentrate.

For plant material comprising spinach/broccoli/kale, decarboxylation may commence at around 60-70° C. and the liquid concentrate is controlled to be between 90-100° C. for a predetermined period of time.

The liquid concentrate may be cleaned by further reduction to an oil in an oven. The further reduction may also includes decarboxylation of the liquid concentrate. Decarboxylation may be activated with controlled laser temperature to avoid the liquid concentrate from being heated above a temperature of around 79° C. until all the solvent present in the liquid concentrate is reduced and evaporated.

### BRIEF DESCRIPTION OF THE DRAWINGS

The invention may be better understood from the following non-limiting description of preferred embodiments, in which:

FIG. 1 is a flow chart depicting the steps associated with forming the solution of the present invention in accordance with one embodiment thereof.

### DETAILED DESCRIPTION OF THE DRAWINGS

Preferred features of the present invention will now be described with particular reference to the accompanying drawings. However, it is to be understood that the features illustrated in and described with reference to the drawings are not to be construed as limiting on the scope of the invention.

The present process of the present invention will be described below in relation to the extraction of ATP rich oil from parts of any given plant material. However, it will be appreciated that the process could be employed to extract ATP and other constituents and chemicals from a variety of chlorophyl based plant materials as would be appreciated by those skilled in the art.

The present invention can also be applied to the extraction of CBD oil. Cannabidiol (CBD) is a chemical compound present in the Cannabis plant, also known as marijuana or hemp. Over 400+ chemicals, underneath CBD (cannabidol) and THC (delta-9-tetrahydrocannabinol), have been found in the Cannabis plant, with THC being the most well known due to its psychoactive effects. However CBD is also present in large volume and CBD has been found to have a variety of medicinal effects, ranging from pain management through to reducing anxiety. function Cannabinoids generally by activating endocannabinoid receptors (CB1 and CB2) and ion channels in cells within the endocannabinoid system. The endocannabinoid system extends throughout the human body but is most abundant in the brain and immune system and has functions relating to control of pain, memory, movement, appetite, metabolism, immunity and cardiopulmonary function. The manner in which cannabinoids, such as CBD, can be used to control and improve such functions, is still under investigation by a variety of medical research bodies.

The amount of CBD that can be extracted from Cannabis plants may vary between plants. Different strains of plants may be selected based on the cannabinoid levels. Typically, extraction of CBD oil requires harvesting parts of the plant and treating the plant to separate the cannabinoids from the plant parts to form a liquid extract. The liquid extract is then treated to form a concentrated and purified CBD oil that may be used directly for a variety of treatments, as required. A problem with most existing processes for extracting the CBD oil is that they fail to produce adenosine triphosphate (ATP) to appropriate levels to facilitate optimal use of the oil in medical applications. Thus, the present invention can be applied to such processes for extracting CBD to enhance the level of ATP present in the CBD oil.

Thus, the present invention can be applied to extract ATP from a variety of chlorophyl based plant materials not just for the CBD oil but for any plant extract.

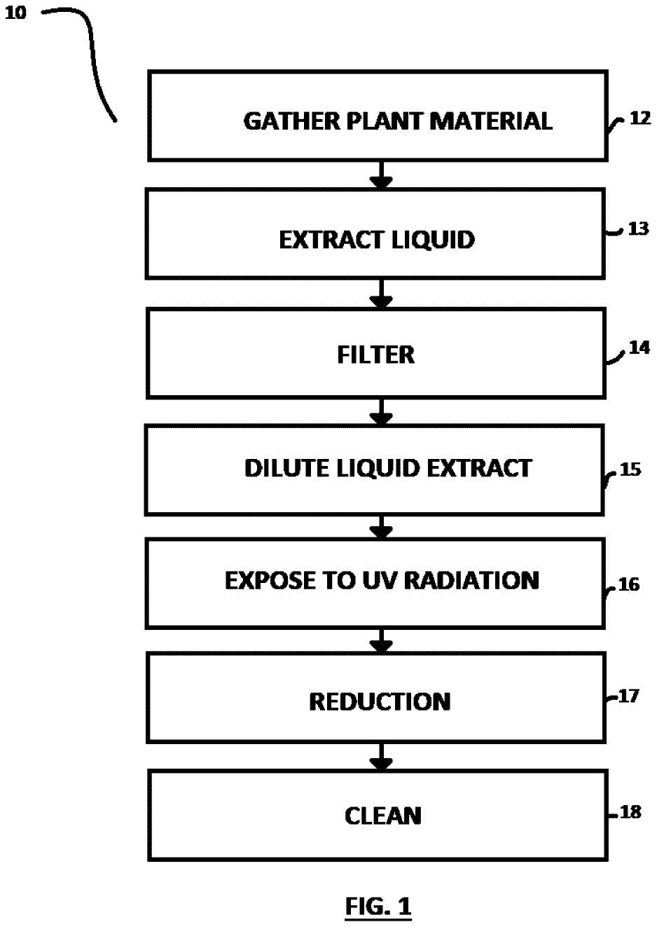

Referring to FIG. 1, an extraction process 10 in accordance with one embodiment of the present invention is shown.

In step 12, parts of a plant, such as a cannabis plant, a spinach plant, or indeed any green plant or autotroph, are harvested. The parts may include bulk flowers, buds, leafs as well as stems and other parts of the plant. The parts may be taken from a single plant species or from a variety of plant species. Upon collection the plant material is dried or dehydrated to remove moisture from the collected plant material

In step 13, a liquid extract is taken from the plant parts through the use of a solvent, such as propanol/alcohol based chemical. In this step the propanol will be applied to the plant material such that the plant material will saturate/soak in the propanol for a predetermined time period, such as at least 24-48 hours. At the end of this soaking period, a propanol based extract will be formed and this extract is then filtered to remove excess plant material and separated. The excess plant material may then be further soaked in propanol to obtain any residual extract present in the plant material. The liquid extract produced on the primary soak will typically be in the form of a dark green liquid.

In step 14, following the extraction process, the extracted liquid material will be filtered to remove fine plant material present therein. This can be achieved by passing the liquid extract through a sieve and then through one or more filter mediums, such as filter paper.

In step 15, an optional step is provided where the liquid extract is diluted with a solvent, such as propanol, but could include any number of solvents, including water, at a desired dilution rate. In a preferred embodiment the liquid extract may be diluted at a level of 1 parts liquid extract to 2 parts propanol, although other dilution rates are also proposed. The resultant liquid extract may then have a lighter green colour for further processing in step 16.

In step 16 the diluted liquid extract is exposed to UV light for a predetermined time period. This may be achieved by placing the diluted liquid in a glass receptacle and exposing the receptacle to natural UV light or a UV lamp whereby the UV light is able to irradiate the diluted liquid extract present therein. The UV light functions to take Chlorophyl and produces Adenosine Triphosphate (ATP) within the liquid extract. The exposure time required will vary depending on the time of year and other environmental conditions. Whilst natural UV light is preferred, lamp generated UV light is also envisaged. The exposure time proportional to the mw/cm2 of UV energy required per litre in a glass jar.

Following the UV exposure step 16, the liquid extract undergoes a reduction step 17, whereby the liquid extract is poured into a still, and reduced using an induction plate, avoiding exposure of the liquid extract to direct flame. The heat applied by the induction plate is able to safely reduce the liquid extract down to a liquid concentrate. For liquid extract taken from plant material such as spinach/broccoli/kale, decarboxylation can begin at around 60-70° C. and may be controlled to be between 90-100° C. for a predetermined period of time. For other material such as cannabinoids, decarboxylation may commence at 100° C. and the liquid concentrate is controlled to be between 100-135° C. for a predetermined period of time.

In step 18, the liquid concentrate is then cleaned by further reduction to an oil in an oven environment. The reduction step typically includes decarboxylation of the liquid extract to activate the desired compounds in the plant material. Decarboxylation can also or alternatively be activated with controlled laser temperature for a more refined outcome. In this regard, the Propanol acts as a heat blanket during this further reduction process, and will not allow the substance to pass a temperature of more than 79° C. until all the propanol present in the liquid is reduced and evaporated.

As the liquid condenses, further minute plant materials may fall out of the liquid solution to settle at the bottom of the bowls of liquid concentrate. At about 50-60% reduction in the oven, these bowls are then poured into each other, leaving a small residue of plant material which is then cleaned out with fresh propanol to produce a clean bowl. Once this liquid material is returned to the concentrate, the concentrate is effectively pure oil, and propanol, and can be reduced further to an oil at the required oven temperature. In this regard, by applying an oven temperature of no more than 100-135° C. any risk of burning the ATP enriched oil is avoided, as this occurs at these temperatures. The propanol will eventually evaporate, leaving a pure oil that will start to bubble as the temperature approaches 100° C.-115° C. The temperature of the liquid extract will be monitored with a laser to ensure that the desired temperatures are not exceeded. By retaining the liquid extract at a temperature of approximately 100-115 degrees for 12-15 minutes, a high quality result is achieved, resulting in an oil rich in ATP.

It will be appreciated that the liquid extract created using this process includes the base oil materials from the plant. The extraction step may include decarboxylation of the plant material to activate the desired compounds in the plant material. Decarboxylation can occur either at the beginning of the of step 12, by baking the collected plant material, or at the end of the extraction step 12, with controlled laser temperature reduction for a more refined outcome.

The resultant ATP rich oils are able to then be used for a variety of applications. These applications may include the use of the resultant oils in pain management and relief, or for the treatment of immune based dysfunction and mitochondrial dysfunction. The oils can also be used in the treatment of cancer, autism, PTSD, insomnia and many other conditions. ATP provides a critical role in the human body and its functions including immune system memory, and brain function. In one application, the resultant oil may be used in cancer treatments, alone, or in combination with low dose radiation as a means of affecting difficult/resistant cancer cells to chemical interference. The ATP oil of the present invention can provide the immune system with a sample of cells by which immune system memory can be retrained in combination with an immunotherapy agent. This method of application effectively and logically “vaccinates” the patient against their own cancer. The principal behind this treatment is not to destroy cells so as the immune system can start to identify and retrain itself to assist in combination with immune therapy underpinned by an ATP/mitochondrial component.

The above process provides for a unique method of manufacturing plant-based solutions with enhanced ATP properties for medical applications.

Throughout the specification and claims the word “comprise” and its derivatives are intended to have an inclusive rather than exclusive meaning unless the contrary is expressly stated or the context requires otherwise. That is, the word “comprise” and its derivatives will be taken to indicate the inclusion of not only the listed components, steps or features that it directly references, but also other components, steps or features not specifically listed, unless the contrary is expressly stated or the context requires otherwise.

It will be appreciated by those skilled in the art that many modifications and variations may be made to the methods of the invention described herein without departing from the spirit and scope of the invention.

1. A method of manufacturing an ATP enriched plant based extract comprising:
gathering a collection of plant material;
saturating the collection of plant material in a solvent to generate a liquid extract;
filtering the liquid extract to remove solid plant matter therefrom to form a filtered liquid extract;
exposing the filtered liquid extract to UV light;
reducing the filtered liquid extract to form a liquid concentrate; and
cleaning the liquid concentrate to create an ATP enriched oil extract.

2. A method according to claim 1, wherein the step of gathering the collection of plant material comprises drying and/or humidifying the plant material to substantially remove moisture therefrom.

3. A method according to claim 1, wherein prior to exposing the filtered liquid extract to UV light, the filtered liquid extract is diluted to form a diluted liquid extract.

4. A method according to claim 3, wherein the step of diluting the filtered liquid extract comprises adding a solvent to the filtered liquid extract at a ratio of one part liquid extract to two part solvent.

5. A method according to claim 4, wherein the solvent is propanol.

6. A method according to claim 1, wherein the step of exposing the filtered liquid extract to facilitate the generation of adenosine triphosphate within the filtered liquid extract comprises exposing the filtered liquid extract to natural UV light.

7. A method according to claim 1 wherein the step of exposing the filtered liquid extract to facilitate the generation of adenosine triphosphate within the filtered liquid extract comprises exposing the filtered liquid extract to UV light generated by a UV lamp.

8. A method according to claim 6, wherein prior to exposing the filtered liquid extract to UV light, the diluted liquid extract is placed in a glass jar such that the UV light can irradiate the filtered liquid extract contained therein.

9. A method according to claim 1, wherein the step of reducing the filtered liquid extract to form a liquid concentrate comprises placing the filtered liquid extract into a still and reduced the filtered liquid extract using an induction plate.

10. A method according to claim 9, wherein the heat applied by the induction plate is able to safely reduce the filtered liquid extract to the liquid concentrate and the heat applied will cause decarboxylation to occur within the liquid concentrate.

11. A method according to claim 10, wherein for plant material comprising spinach/broccoli/kale, decarboxylation will begin at around 60-70° C. and the liquid concentrate is controlled to be between 90-100° C. for a predetermined period of time.

12. A method according to claim 10, wherein for plant material comprising cannabinoids, decarboxylation will begin at around 100° C. and the liquid concentrate is controlled to be between 100-135° C. for a predetermined period of time.

13. A method according to claim 1, wherein the liquid concentrate is cleaned by further reduction to an oil in an oven.

14. A method according to claim 13, wherein further reduction also includes decarboxylation of the liquid concentrate.

15. A method according to claim 14, wherein decarboxylation is activated with controlled laser temperature to avoid the liquid concentrate from being heated above a temperature of around 79° C. until all the solvent present in the liquid concentrate is reduced and evaporated.
